(19) United States
(12) Patent Application Publication
(10) Pub. No.: US 2023/0390393 A1
(43) Pub. Date: Dec. 7, 2023

(54) CHIMERIC ANTIGEN RECEPTOR COMPRISING AN ANTIGEN BINDING DOMAIN SPECIFIC FOR MSLN HAVING A SPECIFICITY FOR TUMOR CELLS

(71) Applicant: Miltenyi Biotec B.V. & Co KG, Bergisch Gladbach (DE)

(72) Inventors: Olaf HARDT, Bergisch Gladbach (DE); Andreas BOSIO, Bergisch Gladbach (DE)

(73) Assignee: Miltenyi Biotec B.V. & Co KG, Bergisch Gladbach (DE)

(21) Appl. No.: 18/033,335

(22) PCT Filed: Nov. 2, 2021
(86) PCT No.: PCT/EP2021/080310

§ 371 Date: Apr. 22, 2023

(30) Foreign Application Priority Data
Nov. 4, 2020 (EP) 20205657.8

(51) Int. Cl. A61K 39/00; C07K 14/725; C07K 16/30; C07K 16/18; C07K 16/28; C07K 14/705; A61P 35/00; C12N 5/0783

(52) U.S. Cl. CPC A61K 39/464468; C07K 14/7051; C07K 16/3023; C07K 16/18; C07K 16/2803; C07K 16/28; C07K 16/2896; C07K 14/70517; C07K 14/70521; C07K 14/70578; A61K 39/4631; A61K 39/4611; A61P 35/00; C12N 5/0636; C07K 2319/03; C07K 2319/02; C12N 2501/2307; C12N 2501/2315

(57) ABSTRACT

The present invention is directed to a chimeric antigen receptor (CAR), comprising an antigen binding domain specific for MSLN in combination with one or more antigen binding domains specific for an antigen selected from the group consisting of CLA, CD66c, TSPAN8 and CD318; cell populations expressing such CARs and the use of the cell populations for cancer therapy.

### BACKGROUND

The present invention relates to the use of ligands comprising antigen binding domains specific for certain antigens, like chimeric antigen receptors (CAR) and/or engineered cells provided with such ligands for treatment of human cancer.

Cancer is a broad group of diseases involving unregulated cell growth. In cancer, cells divide and grow uncontrollably, forming malignant tumors, and invading nearby parts of the body. The cancer may also spread to more distant parts of the body through the lymphatic system or bloodstream. There are over 200 different known cancers that affect humans. Whereas good treatment options are available for many cancer types, others still represent unmet medical needs.

The technology of chimeric antigen receptor (CAR) may provide a promising approach for adoptive cell immunotherapy for cancer. Commonly, CARs comprise a single chain fragment variable (scFv) of an antibody specific for a tumor associated antigen (TAA) coupled via hinge and transmembrane regions to cytoplasmic domains of T-cell signaling molecules. For example, well known lymphocyte activation moieties include a T-cell costimulatory (e.g. CD28, CD137, OX40, ICOS, and CD27) domain in tandem with a T-cell triggering (e.g. CD3ζ) moiety. The CAR-mediated adoptive immunotherapy allows CAR-grafted cells to directly recognize the TAAs on target tumor cells in a non-HLA-restricted manner.

Paramount for immunotherapy for cancer based on CAR is the selection of antigens specific for the respective tumor cells. Object of the invention was to provide such antigens specific for cancer cells, especially for pancreas cancer cells in order to engineer killer cells which then kill/lyse cancer cells without attacking non-tumor cells.

### SUMMARY OF THE INVENTION

It has been found that a distinct group of cell surface antigens is expressed on several human cancer cells, especially on human pancreas cancer cells, but not or to a lower level on non-malignant cells. Accordingly, these antigens (also referred to as “markers”) can be used to identify and/or mark and/or destroy and/or disable escape mechanisms of such cancer cells via ligands that specifically bind to the markers. Most important, it was found that Mesothelin (MSLN) is one these distinct markers, but a better specifity for cancer cells can be reached in combination with one or more further markers.

Therefore, the invention relates to a chimeric antigen receptors (CAR), comprising an antigen binding domain specific for MSLN in combination with one or more antigen binding domains specific for one or more antigens selected from the group consisting of CLA, CD66c, TSPAN8 and CD318. The antigen binding domains can be present on the same or different chimeric antigen receptor (CAR).

For example, the chimeric antigen receptor (CAR) according to the invention may comprise antigen binding domains specific for MSLN in combination with an antigen binding domain specific for CLA (or any of CD66c, TSPAN8 and CD318).

In another variant, the chimeric antigen receptor (CAR) according to the invention may comprise an antigen binding domain specific for MSLN in combination with an antigen binding domain specific for CLA and CD66c (or any of TSPAN8 and CD318).

Another object of the invention are methods of binding a cancer cell with a chimeric antigen receptor (CAR), comprising an antigen binding domain specific for MSLN in combination with one or more antigen binding domains specific for one or more antigens selected from the group consisting of CLA, CD66c, TSPAN8 and CD318.

Another object of the invention are populations of engineered cells expressing at least one of said chimeric antigen receptors (CAR). The engineered cells may express the antigen binding domains on the same or different chimeric antigen receptor (CAR). Further objects are pharmaceutical compositions comprising the population of engineered cells and/or the use of the population of engineered cells or the pharmaceutical composition for treatment of human cancer.

### BRIEF DESCRIPTION OF THE DRAWINGS

FIG. 1 shows the general structure of a CAR capable of recognizing a specific target

FIG. 2 shows the variants of CAR

FIG. 3 shows the results for Different binders for CD66c, CD318, TSPAN8 and MSLN show all effective specific killing on AsPC1 cells.

FIG. 4 shows the results for different binders for CD66c, CD318, TSPAN8 and MSLN show all effective specific killing on Panc0201 cells.

FIG. 5 shows the results for different binders for CD66c, CD318, TSPAN8 and MSLN show all effective specific killing on Panc0203 cells

### DETAILED DESCRIPTION OF THE INVENTION

In a first embodiment of the invention, the chimeric antigen receptor (CAR) comprises an antigen binding domain specific for MSLN in combination with at least two antigen binding domains specific for at least two different antigens selected from the group consisting of CLA, CD66c, TSPAN8 and CD318. The antigen binding domains can be present on the same or different chimeric antigen receptor (CAR).

In a preferred method of binding a cancer cell, the cancer cell (or population of cancer cells) is bound with a chimeric antigen receptor (CAR) comprising an antigen binding domain specific for MSLN in combination with at least two antigen binding domains specific for at least two different antigens selected from the group consisting of CLA, CD66c, TSPAN8 and CD318.

### Definitions

The term “tumor” is known medically as a neoplasm. Not all tumors are cancerous; benign tumors do not invade neighboring tissues and do not spread throughout the body.

The term “cancer” is known medically as a malignant neoplasm. Cancer is a broad group of diseases involving unregulated cell growth. In cancer, cells (cancerous cells) divide and grow uncontrollably, forming malignant tumors, and invading nearby parts of the body. The cancer may also spread to more distant parts of the body through the lymphatic system or bloodstream.

The term “isolated” means altered or removed from the natural state. For example, an isolated population of cells means an enrichment of such cells and separation from other cells which are normally associated in their naturally occurring state with said isolated cells. An isolated population of cells means a population of substantially purified cells which is a homogenous population of cells.

The terms “specifically binds” or “specific for” with respect to an antigen-binding domain of a ligand like an antibody, of a fragment thereof or of a CAR refer to an antigen-binding domain which recognizes and binds to a specific antigen, but does not substantially recognize or bind other molecules in a sample. An antigen-binding domain that binds specifically to an antigen from one species may bind also to that antigen from another species. This cross-species reactivity is not contrary to the definition of that antigen-binding domain as specific. An antigen-binding domain that specifically binds to an antigen may bind also to different allelic forms of the antigen (allelic variants, splice variants, isoforms etc.). This cross reactivity is not contrary to the definition of that antigen-binding domain as specific.

The terms “engineered cell” and “genetically modified cell” as used herein can be used interchangeably. The terms mean containing and/or expressing a foreign gene or nucleic acid sequence which in turn modifies the genotype or phenotype of the cell or its progeny. Especially, the terms refers to cells, preferentially T cells which are manipulated by recombinant methods well known in the art to express stably or transiently peptides or proteins which are not expressed in these cells in the natural state. For example, T cells are engineered to express an artificial construct such as a chimeric antigen receptor on their cell surface. For example, the sequences encoding the CAR may be delivered into cells using a retroviral or lentiviral vector.

The amino acid sequences given in SEQ ID NO:1-40, respectively (in the one-letter code of amino acids) shall refer to all constellations of the respective amino acid sequence which retains the intended function of the respective amino acid sequence as defined. Therefore, all variants of the amino acid sequences defined in the sequence listings having a sequence identity of at least 70%, or at least 75%, 80%, 85%, 90%, 95%, 97%, 98% or 99% at the amino acid sequence level are included in the scope of the present invention. In the context of the present invention, “sequence identity” may be determined using pairwise alignments using alignments programs for amino acid sequences well known to the art.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are several subsets of T cells, each with a distinct function.

T helper cells (TH cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including TH1, TH2, TH3, TH17, Th9, or TFH, which secrete different cytokines to trigger a different type of immune response. Signaling from the APC directs T cells into particular subtypes.

Cytotoxic T cells (TC cells, or CTLs) destroy infected cells and tumor cells, and are also implicated in transplant rejection. These cells are also known as CD8+ T cells since they express the CD8 glycoprotein at their surface. These cells recognize their targets by binding to antigen associated with MHC class I molecules, which are present on the surface of all nucleated cells.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface molecule CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4+ Treg cells have been described—Foxp3+ Treg cells and Foxp3− Treg cells.

Natural killer T cells (NKT cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d. Once activated, these cells can perform functions ascribed to both Th and Tc cells (i.e., cytokine production and release of cytolytic/cell killing molecules).

Immunotherapy is a medical term defined as the “treatment of disease by inducing, enhancing, or suppressing an immune response”. Immunotherapies designed to elicit or amplify an immune response are classified as activation immunotherapies, while immunotherapies that reduce or suppress are classified as suppression immunotherapies. Cancer immunotherapy as an activating immunotherapy attempts to stimulate the immune system to reject and destroy tumors. Adoptive cell transfer uses cell-based, such as T cell-based cytotoxic responses to attack cancer cells. T cells that have a natural or genetically engineered reactivity to a patient's cancer are generated in vitro and then transferred back into the cancer patient.

The term “biomarker” or “marker” is widespread in the art and may broadly denote a biological molecule and/or a detectable portion thereof (e.g. a nucleic acid, a peptide or a lipid such as a glycolipid) whose qualitative and/or quantitative evaluation in an individual is predictive or informative (e.g., predictive, diagnostic and/or prognostic) with respect to one or more aspects of the individual's phenotype and/or genotype. E.g. the biomarker is predictive or informative with respect to the outcome for chemotherapeutic treatment of a cancer in an individual. A biomarker is expressed (“expression of the biomarker”) if the biomarker is detectable with methods known in the art. Therefore expression of biomarkers encompasses not only expression at nucleic acid level (DNA and/or RNA) and protein level but also expression (presence) of other biological structures on or in the cells such as glycolipids or the activity of a protein.

The term “target” as used herein refers to an antigen or epitope associated with a cell that should be recognized specifically by an antigen binding domain, e.g. an antigen binding domain of an antibody or of a CAR. The antigen or epitope for antibody recognition can be bound to the cell surface but also be secreted, part of the extracellular membrane, or shed from the cell.

The term “antibody” as used herein refers to polyclonal or monoclonal antibodies and fragments thereof, which can be generated by methods well known to the person skilled in the art. The antibody may be of any species, e.g. mice, rat, sheep, human. For therapeutic purposes, if non-human antigen binding fragments are to be used, these can be humanized by any method known in the art. The antibodies may also be modified antibodies (e.g. oligomers, reduced, oxidized and labeled antibodies).

The term “killer cell” as used herein refers to a cell that can kill/lyse another cell, e.g. a cancer cell. Most frequently, T cells, NK cells, dendritic cells and macrophages can be used as killer cells.

The term “engineered killer cell” as used herein refers to a killer cell that is genetically modified to allow for the specific killing of a target cell, e.g. a cell modified with a CAR against a target to kill tumor cell expressing the respective target.

### Chimeric Antigen Receptor (CAR)

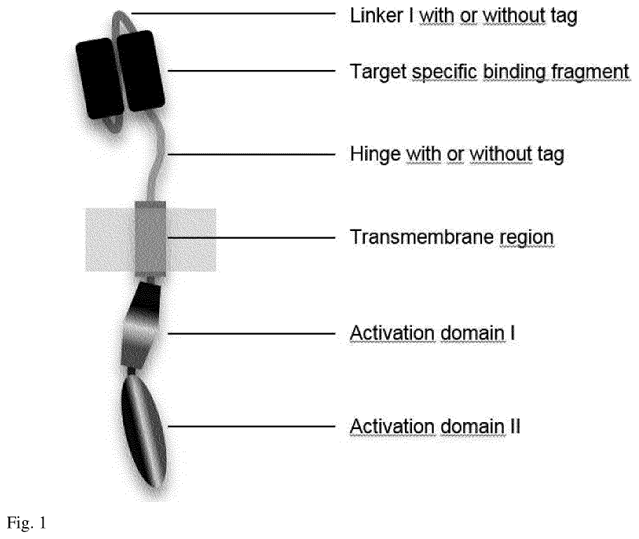

The chimeric antigen receptor (CAR) according to the invention may comprise an antigen binding domain conjugated to a transmembrane domain and/or an intracellular signaling domain, as shown by way of example in FIG. 1.

In yet another embodiment of the invention, the chimeric antigen receptor (CAR) comprises an antigen binding domain, an transmembrane domain and/or an intracellular signaling domain and comprising an antigen binding domain specific for MSLN in combination with one or more antigen binding domains specific for an antigen selected from the group consisting of CLA, CD66c, TSPAN8 and CD318 which are conjugated to the same or a different transmembrane domain and/or intracellular signaling domain.

In another embodiment of the invention, the antigen binding domain of a CAR binds a hapten that is coupled to a polypeptide (“haptenylated” polypeptide), wherein the polypeptide may bind to a tumor associated antigen. Such CARs are for example disclosed in U.S. Pat. No. 9,233,125B2 and are known in the art as “anti-tag” CAR. Similar, the extracellular part of the CAR of the invention may comprise a linker/label epitope (LLE) binding domain as antigen binding domain that binds to a linker/label epitope (LLE) that is part of a target cell binding molecule Such “anti-LLE CARs” are disclosed in the European patent application no. EP16196487.9. Both types of CARs are universal and/or adaptable CAR. Both the hapten(s) and the LLE are “tags” that are coupled directly or indirectly to a polypeptide (the tagged polypeptide), wherein the polypeptide may bind to a tumor associated antigen expressed on the (cell) surface of a target cell.

In this embodiment, the CAR comprises an anti-tag binding region which can bind or binds to a tag which is coupled to an antigen binding domain specific for one or more antigens selected from the group consisting of MSLN, CLA, CD66c, TSPAN8 and CD318.

In another embodiment, the CAR comprises an anti-tag binding region which can bind or binds to a tag which is coupled to an antigen binding domain specific for MSLN in combination with at least two antigen binding domains specific for at least two different antigens selected from the group consisting of CLA, CD66c, TSPAN8 and CD318.

Suitable tags are for example, but not limited to, Biotin, other haptens, FITC or other fluorochrome molecules, FLAG, HIS, YOL MYC, Dextran, FcR, antibody-isotypes, artificially engineered epitopes, FAB or FAB2 binders.

The transmembrane domain of the CAR may comprise a sequence of the transmembrane domains of 4-1BB, CD8 and/or CD28; and the intracellular signaling domain comprises a sequence of the intracellular signaling domains of one or more of CD28, CD137 and CD3zeta.

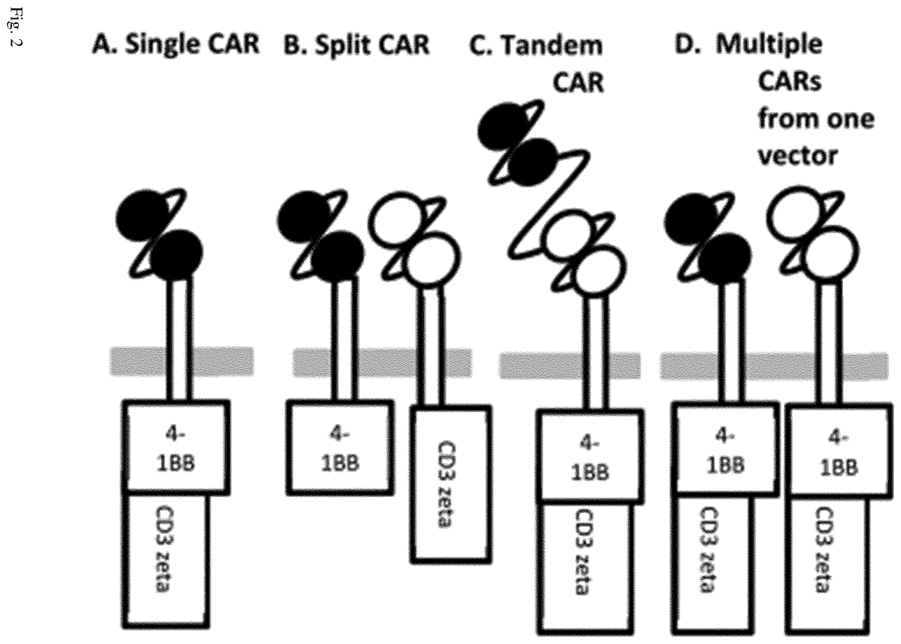

In a second variant of the invention, the chimeric antigen receptor (CAR), comprises an antigen binding domain specific for MSLN in combination with one or more antigen binding domains specific for an antigen selected from the group consisting of CLA, CD66c, TSPAN8 and CD318, wherein the antigen binding domains are conjugated to different transmembrane domains and/or signaling domains. This variant is shown by way of example in FIG. 2 b.

In a third variant of the invention, the chimeric antigen receptor (CAR) comprises an antigen binding domain specific for MSLN in combination with at least two antigen binding domains specific for at least two different antigens selected from the group consisting of CLA, CD66c, TSPAN8 and CD318, wherein the antigen binding domains are conjugated to the same (one) transmembrane domain and signaling domains. This variant is shown by way of example in FIG. 2 c.

In a forth variant of the invention, the chimeric antigen receptor (CAR) comprises an antigen binding domain specific for MSLN in combination with at least two antigen binding domains specific for at least two different antigens selected from the group consisting of CLA, CD66c, TSPAN8 and CD318, wherein the antigen binding domains are conjugated to different transmembrane domains and signaling domains and the antigen binding domains origin from one vector. This variant is shown by way of example in FIG. 2 d.

CLA is the cutaneous lymphocyte-associated antigen (CLA), a specialized glycoform of P-selectin glycoprotein ligand-1 (PSGL-1). It serves as a ligand for selectins, including CD62E (ELAM-1) and CD62L (LECAM-1). CLA is a unique skin-homing receptor and is predominantly found on a minor subset of human T cells that infiltrate the skin. This post-translational modification of PSGL-1 is thought to serve as a mechanism to regulate tissue-specific homing of CD4+ and CD8+ memory/effector T cells from peripheral blood to the skin, which plays an essential role during many inflammatory and certain malignant skin diseases.

In peripheral blood, CLA is not only found on skin-homing memory/effector T cells, but is also found to be expressed on memory/effector B cells, NK cells, blood dendritic cells, and on monocytes. CLA is furthermore found on Langerhans cells in the skin.

Mesothelin is a tumor differentiation antigen that is normally present on the mesothelial cells lining the pleura, peritoneum and pericardium. Mesothelin is upregulated in mesothelioma, and in pancreatic, ovarian and other cancers (Morello A, SadelainM, AdusumilliPS. Mesothelin-Targeted CARs: Driving T Cells to Solid Tumors. Cancer Discov. 2016 Feb;6(2):133-46; U.S. incidence/prevalence).

The antigen binding domain of said CAR may comprise, for example, full length heavy chain, Fab fragments, single chain Fv (scFv) fragments, divalent single chain antibodies or diabodies, each of which are specific for one or more of the target antigens MSLN, CLA, CD66c, TSPAN8 and CD318.

The antigen binding domain of said CAR may comprise the amino acid sequences of SEQ ID NO:1 and SEQ ID NO:2. The relevant sites causing specificity for antigen binding are the CDRs according to the IMGT (the international ImMunoGeneTics information system for immunoglobulins or antibodies) definition which are underlined in the sequence. The antigen binding domain of said CAR may comprise a scFv comprising the amino acid sequence of SEQ ID NO:17 or SEQ ID NO:18.

The present invention also encompasses nucleic acids (DNA or RNA) constructs comprising sequences encoding for amino acids sequences of a CAR specific for the disclosed markers.

In one embodiment of the invention a DNA construct (vector, plasmid) is generated encoding for a CAR specific for the disclosed markers. A nucleic acid sequence encoding for an antigen binding domain specific for the disclosed markers is fused at least to a nucleic acid sequence encoding a transmembrane domain and subsequent a nucleic acid sequence encoding a intracellular domain. The construction of such expression vectors can be performed by recombinant methods well known in the art. Alternatively, the nucleic acid sequences can be produced synthetically.

Alternatively, the CAR may be composed of further parts such as a linker and/or hinge and/or may be composed as di- or multi-chain CAR as described below.

As shown in general in FIGS. 1 and 2, a CAR may comprise an extracellular domain comprising the antigen binding domain, a transmembrane domain and an intracellular signaling domain. The extracellular domain may be linked to the transmembrane domain by a linker. The extracellular domain may also be linked to a signal peptide.

A “signal peptide” refers to a peptide sequence that directs the transport and localization of the protein within a cell, e.g. to a certain cell organelle (such as the endoplasmic reticulum) and/or the cell surface.

An “antigen binding domain” refers to the region of the CAR that specifically binds to an antigen (and thereby is able to target a cell containing an antigen). The CARs of the invention may comprise one or more antigen binding domains. Generally, the antigen binding domain on the CAR are extracellular. The antigen binding domain may comprise an antibody or a fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors can be used as an antigen binding domain. Often the antigen binding domain is a scFv. Normally, in a scFv the variable portions of an immunoglobulin heavy chain and light chain are fused by a flexible linker to form a scFv. Such a linker may be for example the “(G4/S1)3-linker”.

In some instances, it is beneficial for the antigen binding domain to be derived from the same species in which the CAR will be used in. For example, if it is planned to use it therapeutically in humans, it may be beneficial for the antigen binding domain of the CAR to comprise a human or humanized antibody or fragment thereof. Human or humanized antibodies or fragments thereof can be made by a variety of methods well known in the art.

“Spacer” or “hinge” as used herein refers to the hydrophilic region which is between the antigen binding domain and the transmembrane domain. The CARs of the invention may comprise an extracellular spacer domain but is it also possible to pass such a spacer. The spacer may include Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge.

The transmembrane domain of the CAR can be derived from any desired natural or synthetic source for such domain. If the source is natural the domain may be derived from any membrane-bound or transmembrane protein. The transmembrane domain may be derived for example from CD8alpha or CD28.

The cytoplasmic domain or the intracellular signaling domain of the CAR of the invention is responsible for activation of at least one of the normal effector functions of the immune cell in which the CAR is expressed. “Effector function” means a specialized function of a cell, e.g. in a T cell an effector function may be cytolytic activity or helper activity including the secretion of cytokines. The intracellular signaling domain refers to the part of a protein which transduces the effector function signal and directs the cell expressing the CAR of the invention to perform a specialized function. The intracellular signaling domain may include any complete or truncated part of the intracellular signaling domain of a given protein sufficient to transduce the effector function signal.

Prominent examples of intracellular signaling domains for use in the CARs include the cytoplasmic sequences of the T cell receptor (TCR) and co-receptors that act in concert to initiate signal transduction following antigen receptor engagement.

Generally, T cell activation can be mediated by two distinct classes of cytoplasmic signaling sequence, firstly those that initiate antigen-dependent primary activation through the TCR (primary cytoplasmic signaling sequences) and secondly those that act in an antigen-independent manner to provide a secondary or co- stimulatory signal (secondary cytoplasmic signaling sequences).

Primary cytoplasmic signaling sequences that act in a stimulatory manner may contain ITAMs (immunoreceptor tyrosine-based activation motifs signaling motifs).

Examples of ITAM containing primary cytoplasmic signaling sequences often used in chimeric antigen receptor (CAR) derived from TCR zeta, FcR gamma, FcR beta, CD3 gamma , CD3 delta , CD3 epsilon, CD5, CD22, CD79a, CD79b, and CD66d.

The cytoplasmic domain of the CAR can be designed to comprise the CD3-zeta signaling domain by itself or combined with any other desired cytoplasmic domain(s). The cytoplasmic domain of the CAR can comprise a CD3 zeta chain portion and a costimulatory signaling region. The costimulatory signaling region refers to a part of the CAR comprising the intracellular domain of a costimulatory molecule. A costimulatory molecule is a cell surface molecule other than an antigen receptor or their ligands that is required for an efficient response of lymphocytes to an antigen. Examples for costimulatory molecule are CD27, CD28, 4-1BB (CD137), OX40, CD30, CD40, PD-1, ICOS, lymphocyte function-associated antigen- 1 (LFA-1), CD2, CD7, LIGHT, NKG2C, B7-H3.

The cytoplasmic signaling sequences within the cytoplasmic signaling part of the CAR may be linked to each other in a random or specified order. A short oligo- or polypeptide linker, which is preferably between 2 and 10 amino acids in length, may form the linkage. A prominent linker is the glycine- serine doublet.

As an example, the cytoplasmic domain may comprise the signaling domain of CD3-zeta and the signaling domain of CD28. In another example the cytoplasmic domain may comprise the signaling domain of CD3-zeta and the signaling domain of CD27. In an further example, the cytoplasmic domain may comprise the signaling domain of CD3-zeta, the signaling domain of CD28, and the signaling domain of CD27.

### Engineered Cells Expressing the CAR

In another embodiment of the invention, the engineered cell (or a population thereof), expresses least one CAR comprising an antigen binding domain specific for MSLN in combination with at least one antigen binding domains specific for at least on antigen selected from the group consisting of CLA, CD66c, TSPAN8 and CD318.

In a preferred embodiment, the engineered cell (or a population thereof), expresses least one CAR comprising an antigen binding domain specific for MSLN in combination with at least two antigen binding domains specific for at least two different antigens selected from the group consisting of CLA, CD66c, TSPAN8 and CD318.

The CAR of the invention or cells expressing the CAR of the invention may comprise an antigen binding domain, a transmembrane domain and an intracellular signaling domain, wherein the intracellular signaling domain is triggered by binding of the antigen binding domain specific for MSLN and/or one antigen binding domain specific for an antigen selected from the group consisting of CLA, CD66c, TSPAN8 and CD318 to the appropriate antigens.

The population of engineered cells may consist of T cells, macrophages or NK cells. The population of engineered cells may be are expanded to an therapeutically effective amount of cells before use in said immunotherapy.

To generate cells expressing the one or more CAR of the invention (including the variants), a DNA construct encoding the CAR of the invention can be transfected or transduced into a host cell by methods well known in the art (e.g. viral-based systems, physical methods, biological methods, chemical methods). Regardless of the methods used to integrate the nucleic acid encoding the CAR of the invention in the host cell, as a result the host cell expresses a CAR which is specific for the markers as disclosed.

In one embodiment of the invention, the engineered cells are isolated (enriched or separated) after the transfection/transduction process for generating such an engineered cell from non-transfected/transduced cells by methods well known in the art, e.g. fluorescent based separating technologies such as FACS® or magnetic cell separation methods such as MACS®.

In another embodiment of the invention a source of immune cells, preferentially T cells is obtained from a subject. Immune cells, preferentially T cells can be obtained from a variety of sources such as peripheral blood mononuclear cells (PMBCs), bone marrow, lymph node tissue, cord blood or thymus tissue. For enrichment of these cells methods well known in the art can be used such as centrifugation through a Ficoll™ or PERCOLL™ gradient or positive/negative selection techniques such as fluorescent sorting (e.g. FACS sort) or magnetic sorting (e.g. MACS®).

For example, T cells of a blood sample of a subject are magnetically labelled, for example with a magnetic bead coupled to antibodies specific for CD4 and for CD8 or alternatively CD62L, respectively, washed, magnetically enriched and collected. Then these T cells may be engineered to express the antigens as disclosed or the preferred combination of antigens on their cell surface.

In one embodiment of the invention the isolated/enriched engineered T cells expressing an antigens as disclosed or the preferred combination of antigens prior or after genetic modification can be activated and expanded to increase amount of engineered T cells generally using methods well known in the art, for example polyclonal stimulation with anti-CD3/anti-CD28 beads or anti-CD3/anti-CD28 nanomatrices (as disclosed in EP2711418A1). Preferentially, said amount of engineered T cells is increased to a therapeutic effective amount.

In one embodiment of the invention a cell expressing the CAR of the invention is generated. The RNA encoding the CAR of the invention can be transfected or transduced into a host cell by methods well known in the art (e.g. viral-based systems, physical methods, biological methods, chemical methods). In general, such an “RNA-engineered cell” is disclosed in detail in WO2013/040557. Regardless of the methods used to integrate the RNA encoding the CAR of the invention in the host cell, as a result the host cell expresses a CAR which is specific for an antigen as disclosed or the preferred combination of antigens. Using “RNA-engineered cells” lead to the fact that the CAR is expressed for a limited time in the cell (transient expression).

In one embodiment of the invention, the engineered cells are generated automatically in a closed cell culture system. Such process may comprise the steps:
a) providing a cell sample
b) preparation of the cell sample by centrifugation
c) magnetic separation of the cell, preferentially T cells, T cell subsets or T cell progenitors
d) activation of the enriched cells, preferentially T cells, T cell subsets or T cell progenitors using modulatory agents
e) genetically modifying the cells, preferentially T cells, T cell subsets or T cell progenitors to express one or more CARs as disclosed or the preferred combination of CARs/antigens
f) expansion of the genetically modified T cells, T cell subsets or T cell progenitors
in a cultivation chamber
g) washing of the cultured cells, preferentially T cells, T cell subsets or T cell progenitors.

All these steps may be performed in a closed and sterile system.

The process is especially suited for preparing gene modified cells, preferentially T cells, T cell subsets or T cell progenitors wherein the enriched cells, preferentially T cells, T cell subsets or T cell progenitors are gene modified by using viral and/or non-viral vectors. Any of these steps may be multiplied, omitted or may occur in a different order. In a variant of the invention, the modulatory agents are selected from agonistic antibodies and/or cytokines.

As closed and sterile system for cell modification, the fully automated cell processing device CliniMACS Prodigy® and associated tubing sets (Miltenyi Biotec B.V. & Co. KG, Germany) may be used (WO2009/072003). This closed system meets the requirements of GMP-grade processing of almost any kind of cellular products and may allow reducing clean room requirements, improve technology transfer and harmonization of cell manufacturing processes. It has been developed to fully automate and standardize the manufacturing process of cellular therapeutic agents. The instrument can perform sample loading, cell washing, density-based cell separations including erythrocyte reduction and plasma harvesting, magnetic separation, cell activation, cell modification (transduction), cell culture, and final product formulation.

Thus a flexible integration of process modules (“steps”) is enabled in a closed, automated and safe GMP compliant workflow reproducing a complex desired biological process.

In one embodiment of the invention, the genetically modified cells express one of the targets. To circumvent killing among the genetically modified cell population, this target is temporarily or permanently knocked down or knocked out on the killer cells. Temporal or permanent knock down or knock out of expression can be induced by methods well known in the art, such as siRNA for temporal knock down or the CRISPR system for permanent knock out. To inhibit target expression using these methods, this can be achieved by directly targeting the whole gene encoding for the target, parts of the gene, e.g. specific exons, the promotor region, or controlling genes, such as transcription factors. In the case of target structures representing glycostructures, such as CLA, this can also be achieved by altering the glycosylation site on the backbone protein or one or more of the enzymes catalyzing the glycosylation.

### Methods of Use

Another embodiment of the invention is a method of binding a cancer cell with a CAR comprising an antigen binding domain specific for MSLN in combination with an antigen binding domain specific for an antigen selected from the group consisting of CLA, CD66c, TSPAN8 and CD318.

In a variant thereof another embodiment of the invention, a cancer cell is bound with a CAR comprising an antigen binding domain specific for MSLN in combination with at least two antigen binding domains specific for at least two different antigens selected from the group consisting of CLA, CD66c, TSPAN8 and CD318.

The ligands according to the invention may be used in combination with agents, which bind to the antigen and affect the viability of the cancerous cell expressing this antigen, preferentially kill the cancerous cell. Examples of such agents are oncolytic viruses, BiTEs®, ADCCs and immunotoxins.

An oncolytic virus is a virus that preferentially infects and kills cancer cells. As the infected cancer cells are destroyed by lysis, they release new infectious virus particles to help destroy the remaining tumor. Oncolytic viruses are thought not only to cause direct destruction of the tumor cells, but also to stimulate host anti-tumor immune responses. Specific targeting (e.g. targeting/ligation to the antigens as disclosed) involves modifying the viral coat proteins to target tumor cells (e.g. with antigen binding domain specific for antigens as disclosed) while reducing entry to non-tumor cells.

Bi-specific T-cell engagers (BiTEs®) are a class of artificial bispecific monoclonal antibodies that are investigated for the use as anti-cancer drugs. They direct a host's immune system, more specifically the T cells' cytotoxic activity, against cancer cells. BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to a tumor cell via a tumor specific molecule. Like other bispecific antibodies, and unlike ordinary monoclonal antibodies, BiTEs® form a link between T cells and tumor cells. This causes T cells to exert cytotoxic activity on tumor cells by producing proteins like perforin and granzymes, independently of the presence of MHC I or co-stimulatory molecules. These proteins enter tumor cells and initiate the cell's apoptosis. This action mimics physiological processes observed during T cell attacks against tumor cells.

Antibody-dependent cell-mediated cytotoxicity (ADCC) is a mechanism of attack by the immune system that requires the presence of antibodies bound to the surface of target cells. Antibodies are formed of a binding region (Fab), which binds to the target antigen and the Fc region that can be detected by immune cells via Fc receptors on their surface. These Fc receptors are found on the surface of many cells of the immune system, including natural killer cells. When a natural killer cell encounter cells coated with antibodies, the Fc regions interact with their Fc receptors, leading to the release of perforin and granzyme B. These two chemicals lead to the tumor cell initiating programmed cell death (apoptosis). Antibodies known to induce this method of cell killing include Rituximab, Ofatumumab, Trastuzumab, Cetuximab and Alemtuzumab. Third generation antibodies that are currently being developed have altered Fc regions that have higher affinity for a specific type of Fc receptor, FcγRIIIA, which can increase the rate of ADCC dramatically.

An immunotoxin is a human-made protein that consists of a targeting portion linked to a toxin. When the protein binds to that cell, it is taken in through endocytosis, and the toxin kills the cell. These chimeric proteins are usually made of a modified antibody or antibody fragment, attached to a fragment of a toxin. The “targeting portion” is composed of the Fv portion of an antibody that binds specifically to an antigen expressed by a cell, preferably by a specific cell type. The toxin is usually a cytotoxic protein derived from a bacterial or plant protein, from which the natural binding domain has been removed so that the Fv directs the toxin to the antigen on the target cell.

### Pharmaceutical Composition

Another object of the invention is a pharmaceutical composition comprising a population of engineered cells expressing a CAR as already disclosed, optionally with a pharmaceutical acceptable carrier like Composol or NaCl solution.

### Use for Treatment of Cancer

The population of engineered cells as disclosed and/or the pharmaceutical composition comprising the population of engineered cells may be used in a method for treatment of human cancer with cells expressing the disclosed target molecules, especially of human pancreas cancer.

The pharmaceutical composition comprises preferable a population of engineered cells expressing a CAR as already disclosed. In a variant of the invention, the pharmaceutical composition is used in combination with a chemotherapeutic, radiation, or immunomodulatory agent for treatment of cancer.

The cancer to be treated may include hematopoietic cancer, myelodysplastic syndrome, pancreatic cancer, head and neck cancer, cutaneous tumors, minimal residual disease (MRD) in acute lymphoblastic leukemia (ALL), acute myeloid leukemia (AML), lung cancer, breast cancer, ovarian cancer, prostate cancer, colon cancer, melanoma or other hematological cancer and solid tumors, or any combination thereof. In another embodiment, the cancer includes a hematological cancer such as leukemia (e.g., chronic lymphocytic leukemia (CLL), acute lymphocytic leukemia (ALL), acute myeloid leukemia (AML), or chronic myelogenous leukemia (CML), lymphoma (e.g., mantle cell lymphoma, non-Hodgkin's lymphoma or Hodgkin's lymphoma) or multiple myeloma, or any combination thereof. Furthermore, the cancer may include an adult carcinoma comprising coral and pharynx cancer (tongue, mouth, pharynx, head and neck), digestive system cancers (esophagus, stomach, small intestine, colon, rectum, anus, liver, intrahepatic bile duct, gallbladder, pancreas), respiratory system cancers (larynx, lung and bronchus), bones and joint cancers, soft tissue cancers, skin cancers (melanoma, basal and squamous cell carcinoma), pediatric tumors (neuroblastoma, rhabdomyosarcoma, osteosarcoma, Ewing's sarcoma), tumors of the central nervous system (brain, astrocytoma, glioblastoma, glioma), and cancers of the breast, the genital system (uterine cervix, uterine corpus, ovary, vulva, vagina, prostate, testis, penis, endometrium), the urinary system (urinary bladder, kidney and renal pelvis, ureter), the eye and orbit, the endocrine system (thyroid), and the brain and other nervous system, or any combination thereof.

The treatment of cancer may encompass any method which involves at least one antigen as disclosed or any combination of antigens as disclosed as target molecule. Such methods may be e.g. treatment with agents which bind to the antigen and affect the viability of the cancerous cell expressing this antigen, preferentially kill the cancerous cell. Examples are oncolytic viruses, BiTEs®, ADCCs and immunotoxins as already disclosed.

For the treatment, immune cells, e.g. T cells of a subject may be isolated. The subject may suffer from said cancer or may be a healthy subject. These cells are genetically modified in vitro or in vivo to express one or more CARs of the invention. These engineered cells may be activated and expanded in vitro or in vivo. In a cellular therapy these engineered cells may be infused to a recipient in need thereof. These cells may be a pharmaceutical composition (said cell plus pharmaceutical acceptable carrier). The infused cells are able to kill (or at least stop growth of) cancerous cells expressing one or more of the disclosed antigens in the recipient. The recipient may be the same subject from which the cells was obtained (autologous cell therapy) or may be from another subject of the same species (allogeneic cell therapy).

In one embodiment of the invention the subject suffering from pancreas cancer may be treated with the pharmaceutical composition of the invention together with an immunomodulatory agent, such as but not limited to Rapamycin or agents blocking PD-1/PD-L1 or CTLA4 signaling.

In one embodiment of the invention, due to the fact that the cancerous cells expressing one or more of the disclosed antigens may be only a subpopulation of the cancerous cells of the subject the subject may be treated additionally with chemotherapy or radiotherapy. Chemotherapeutic and radiation agents suited to treat cancers are well known in the art.

In one embodiment of the invention the CAR expressing cells are applied to a subject suffering from cancer, especially pancreas cancer as cellular therapy as disclosed above but in combination with a second activating CAR, which is also expressed on the same engineered cells, recognizing an additional epitope on the cancerous cells expressing one or more of the disclosed antigens to increase the specificity of the engineered cells expressing both CARs. This epitope can be membrane bound, part of the extracellular matrix, or a soluble component.

In one embodiment of the invention the CAR expressing cells are applied to a subject suffering from cancer as cellular therapy as disclosed above but in combination with a second, inhibitory CAR, which is also expressed on the same engineered cells, recognizing an additional epitope to increase the specificity of the engineered cells expressing both CARs. This epitope can be membrane bound, part of the extracellular matrix, or a soluble component.

The immune cells, preferentially T cells engineered to express one or more of the disclosed antigens may be administered either alone, or as a pharmaceutical composition in combination with diluents and/or with other components such as IL-2 or other cytokines or cell populations. Briefly, pharmaceutical compositions of the present invention may comprise a cell population of genetically modified cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins;
polypeptides or amino acids such as glycine; antioxidants; chelating agents such as EDTA or glutathione; adjuvants (e.g., aluminum hydroxide); and preservatives.

Preferentially, the compositions of the present invention are formulated for intravenous administration. The administration of cell compositions to the subject may be carried out in any convenient manner known in the art.

Pharmaceutical compositions of the present invention may be administered in a manner appropriate to the disease to be treated. Appropriate dosages may be determined by clinical trials. But the quantity and frequency of administration will also be determined and influenced by such factors as the condition of the patient, and the type and severity of the patient's disease.

A pharmaceutical composition comprising the immune cells, preferentially T cells disclosed herein may be administered at a dosage of 10⁴ to 10⁹ cells/kg body weight, preferably 10⁵ to 10⁶ cells/kg body weight. The cell compositions may also be administered several times at these dosages. The compositions of cells may be injected directly into a tumor, lymph node, or site of infection.

SEQUENCES
CLA V_{H}
SEQ ID NO: 1
EVQLVESGGGLVQPGNSLKLSCSASGFTFSSYGMHWIRQAPGEGLDWVAYISSSSGT
VYADAVKARFTISRDNAKNTLYLQLNSLKSEDTAIYYCARAQNWDLFDYWGQGVM
VTVSS
CLA V_{L}
SEQ ID NO: 2
QIMLTQQAESLWISPGERVSITCRASQSLLYTDGKHYLSWYQQKPGQTTKALIYHAS
VRTDGVPTRFIGSGSGTEFTLSIEHVQPEDFAIYYCLQTLKSPFTFGSGTKLEIK
CD142 V_{H}
SEQ ID NO: 3
QVQLKQSGPGLVQPSQSLSITCTVSGFSLSNYGVHWVRQSPGKGLEWLGVIWSGGST
DYNVAFISRLIITKDNSKSQVFLKMNSLQADDTAIYFCARTTGSVFNAMDHWGQGTS
VTVSS
CD142 V_{L}
SEQ ID NO: 4
QIVLTQSPALMSASPGEKVTMTCSASSSVTYMYWYQQKPRSSPKPWIYLTSNLASGV
PARFSGSGSGTSYSLTISSVEAEDAATYYCQQWSSNPLTFGAGTKLELK
CD73 V_{H}
SEQ ID NO: 5
EVQLQQSGAELVKPGASVKLSCTASGFNIKDTYIHWVKQRPEQGLEWIGRIDPATGN
TEYDPKFQGKATITADTSSNTAYLHLSSLTSEDTAVYYCARGYYGSSYPPWFAYWGQ
GTLVTVSA
CD73 V_{L}
SEQ ID NO: 6
DIVMTQSHKFMSTSVGDRVSITCKASQDVGSAVAWYQQKPGQSPKLLIYWASTRHT
GVPDRFTGSGSGTDFTLTISNVQSEDLADYFCQQYSSYPLTFGAGTKLELK
CD49c V_{H}
SEQ ID NO: 7
EVQLQQSGAELVKPGASVKLSCTASGFNIKDTYMHWVKQRPEQGLEWIGRIDPANG
HTKYDPKFQGKATITADTSSNAAYLQLNSLTSEDTAVYYCARRVAYAMDYWGQGT
SVTVSS
CD49c V_{L}
SEQ ID NO: 8
ENVLTQSPAIMSASPGEKVTMTCSASSSVTYMHWYQQKSSTSPKLWIYDTSKLASGV
PGRFSGSGSGNSYSLTISSMEAEDVATYCCFQGSGYPLTFGGGTKLEIK
CD66c V_{H}
SEQ ID NO: 9
QVTLKESGPGILKPSQTLSLTCSFSGFSLSTSGMGVGWIRQPSGKSLEWLAHIWWNDE
RYYNPSLKNQLTISKDTSRNQVFLKITSVDTADTATYYCARSPRGYFDYWGHGTTLT
VSS
CD66c V_{L}
SEQ ID NO: 10
DIVMTQSQKFMSTSVGDRVSVTCKASQNVVTNVAWYQQTPGQSPKALIYSASYRYS
GVPDRFSGSGSGTDFTLTISNVQSGDLAEYFCQQYNSYPLTFGAGTKLELK
CD104 V_{H}
SEQ ID NO: 11
QVNLLQSGAALVKPGASVKLSCKASGYTFTDYYIFWVKQSHGKSLEWIGYINPNSGS
TNYNEKFKRKATLSVDKSTNTAYMELSRLTSEDSATYYCTRRAYYGYNPFDYWGQG
VMVTVSS
CD104 V_{L}
SEQ ID NO: 12
DIQMTQTPSSMPASLGERVTISCRASRGINNYLSWYQQNLDGTIKPLIYYTSNLQSGVP
SRFSGSGSGTDYSLTISSLEPEDFAMYYCQQYDSSPWTFGGGTKLELK
CD318 V_{H}
SEQ ID NO: 13
EVQLQQSGAELVRPGALVKLSCKASGFNIKDYYIHWVKQRPEQGLEWIGWIDPENG
HTIYDPKFQGKASITADTSSNTAYLQLSSLTSEDTAVYYCARLTGTTYAMDYWGQGT
SVTVSS
CD318 V_{L}
SEQ ID NO: 14
DIVMTQSHKFMSTSVGDRVSITCKASQDVSTAVAWYQQKSGQSPKLLIYWASTRHT
GVPDRFTGSGSGTDYTLTISSVQAEDLALYYCQQHYSTPYTFGGGTKLEIK
TSPAN8 V_{H}
SEQ ID NO: 15
EVKLLESGGGLVQPGGSMRLSCAASGFTFTDFYMNWIRQPAGKAPEWLGFIRNKAS
GYTTEYNPSVKGRFTISRDNTQNMLYLQMNTLRAEDTATYYCARAHSYYGYDYFDY
WGQGVMVTVSS
TSPAN8 V_{L}
SEQ ID NO: 16
DIQMTQSPASLSASLEEIVTITCQASQDIGNWLSWYQQKPGKSPQLLIYGATSLADGV
PSRFSGSRSGTQYSLKISRLQVEDIRIYYCLQAYSAPWTFGGGTKLELK
CLA specific scFv VH-linker-VL
SEQ ID NO: 17
EVQLVESGGGLVQPGNSLKLSCSASGFTFSSYGMHWIRQAPGEGLDWVAYISSSSGT
VYADAVKARFTISRDNAKNTLYLQLNSLKSEDTAIYYCARAQNWDLFDYWGQGVM
VTVSSGGGGSGGGGSGGGGSQIMLTQQAESLWISPGERVSITCRASQSLLYTDGKHY
LSWYQQKPGQTTKALIYHASVRTDGVPTRFIGSGSGTEFTLSIEHVQPEDFAIYYCLQT
LKSPFTFGSGTKLEIK
CLA specific scFv VL-linker-VH
SEQ ID NO: 18
QIMLTQQAESLWISPGERVSITCRASQSLLYTDGKHYLSWYQQKPGQTTKALIYHAS
VRTDGVPTRFIGSGSGTEFTLSIEHVQPEDFAIYYCLQTLKSPFTFGSGTKLEIKGGGGS
GGGGSGGGGSEVQLVESGGGLVQPGNSLKLSCSASGFTFSSYGMHWIRQAPGEGLD
WVAYISSSSGTVYADAVKARFTISRDNAKNTLYLQLNSLKSEDTAIYYCARAQNWDL
FDYWGQGVMVTVSS
CD142 specific CAR sequence VH-linker-VL
SEQ ID NO: 19
QVQLKQSGPGLVQPSQSLSITCTVSGFSLSNYGVHWVRQSPGKGLEWLGVIWSGGST
DYNVAFISRLIITKDNSKSQVFLKMNSLQADDTAIYFCARTTGSVFNAMDHWGQGTS
VTVSSGGGGSGGGGSGGGGSQIVLTQSPALMSASPGEKVTMTCSASSSVTYMYWYQ
QKPRSSPKPWIYLTSNLASGVPARFSGSGSGTSYSLTISSVEAEDAATYYCQQWSSNP
LTFGAGTKLELK
CD142 specific CAR sequence VL-linker-VH
SEQ ID NO: 20
QIVLTQSPALMSASPGEKVTMTCSASSSVTYMYWYQQKPRSSPKPWIYLTSNLASGV
PARFSGSGSGTSYSLTISSVEAEDAATYYCQQWSSNPLTFGAGTKLELKGGGGSGGG
GSGGGGSQVQLKQSGPGLVQPSQSLSITCTVSGFSLSNYGVHWVRQSPGKGLEWLGV
IWSGGSTDYNVAFISRLIITKDNSKSQVFLKMNSLQADDTAIYFCARTTGSVFNAMDH
WGQGTSVTVSS
CD73 specific CAR sequence VH-linker-VL
SEQ ID NO: 21
EVQLQQSGAELVKPGASVKLSCTASGFNIKDTYIHWVKQRPEQGLEWIGRIDPATGN
TEYDPKFQGKATITADTSSNTAYLHLSSLTSEDTAVYYCARGYYGSSYPPWFAYWGQ
GTLVTVSAGGGGSGGGGSGGGGSDIVMTQSHKFMSTSVGDRVSITCKASQDVGSAV
AWYQQKPGQSPKLLIYWASTRHTGVPDRFTGSGSGTDFTLTISNVQSEDLADYFCQQ
YSSYPLTFGAGTKLELK
CD73 specific CAR sequence VL-linker-VH
SEQ ID NO: 22
DIVMTQSHKFMSTSVGDRVSITCKASQDVGSAVAWYQQKPGQSPKLLIYWASTRHT
GVPDRFTGSGSGTDFTLTISNVQSEDLADYFCQQYSSYPLTFGAGTKLELKGGGGSGG
GGSGGGGSEVQLQQSGAELVKPGASVKLSCTASGFNIKDTYIHWVKQRPEQGLEWIG
RIDPATGNTEYDPKFQGKATITADTSSNTAYLHLSSLTSEDTAVYYCARGYYGSSYPP
WFAYWGQGTLVTVSA
CD49c specific CAR sequence VH-linker-VL
SEQ ID NO: 23
EVQLQQSGAELVKPGASVKLSCTASGFNIKDTYMHWVKQRPEQGLEWIGRIDPANG
HTKYDPKFQGKATITADTSSNAAYLQLNSLTSEDTAVYYCARRVAYAMDYWGQGT
SVTVSSGGGGSGGGGSGGGGSENVLTQSPAIMSASPGEKVTMTCSASSSVTYMHWY
QQKSSTSPKLWIYDTSKLASGVPGRFSGSGSGNSYSLTISSMEAEDVATYCCFQGSGY
PLTFGGGTKLEIK
CD49c specific CAR sequence VL-linker-VH
SEQ ID NO: 24
ENVLTQSPAIMSASPGEKVTMTCSASSSVTYMHWYQQKSSTSPKLWIYDTSKLASGV
PGRFSGSGSGNSYSLTISSMEAEDVATYCCFQGSGYPLTFGGGTKLEIKGGGGSGGGG
SGGGGSEVQLQQSGAELVKPGASVKLSCTASGFNIKDTYMHWVKQRPEQGLEWIGR
IDPANGHTKYDPKFQGKATITADTSSNAAYLOLNSLTSEDTAVYYCARRVAYAMDY
WGQGTSVTVSS
CD66c specific CAR sequence VH-linker-VL
SEQ ID NO: 25
QVTLKESGPGILKPSQTLSLTCSFSGFSLSTSGMGVGWIRQPSGKSLEWLAHIWWNDE
RYYNPSLKNQLTISKDTSRNQVFLKITSVDTADTATYYCARSPRGYFDYWGHGTTLT
VSSGGGGSGGGGSGGGGSDIVMTQSQKFMSTSVGDRVSVTCKASQNVVTNVAWYQ
QTPGQSPKALIYSASYRYSGVPDRFSGSGSGTDFTLTISNVQSGDLAEYFCQQYNSYP
LTFGAGTKLELK
CD66c specific CAR sequence VL-linker-VH
SEQ ID NO: 26
DIVMTQSQKFMSTSVGDRVSVTCKASQNVVTNVAWYQQTPGQSPKALIYSASYRYS
GVPDRFSGSGSGTDFTLTISNVQSGDLAEYFCQQYNSYPLTFGAGTKLELKGGGGSG
GGGSGGGGSQVTLKESGPGILKPSQTLSLTCSFSGFSLSTSGMGVGWIRQPSGKSLEW
LAHIWWNDERYYNPSLKNQLTISKDTSRNQVFLKITSVDTADTATYYCARSPRGYFD
YWGHGTTLTVSS
CD104 specific CAR sequence VH-linker-VL
SEQ ID NO: 27
QVNLLQSGAALVKPGASVKLSCKASGYTFTDYYIFWVKQSHGKSLEWIGYINPNSGS
TNYNEKFKRKATLSVDKSTNTAYMELSRLTSEDSATYYCTRRAYYGYNPFDYWGQG
VMVTVSSGGGGSGGGGSGGGGSDIQMTQTPSSMPASLGERVTISCRASRGINNYLSW
YQQNLDGTIKPLIYYTSNLQSGVPSRFSGSGSGTDYSLTISSLEPEDFAMYYCQQYDSS
PWTFGGGTKLELK
CD104 specific CAR sequence VL-linker-VH
SEQ ID NO: 28
DIQMTQTPSSMPASLGERVTISCRASRGINNYLSWYQQNLDGTIKPLIYYTSNLQSGVP
SRFSGSGSGTDYSLTISSLEPEDFAMYYCQQYDSSPWTFGGGTKLELKGGGGSGGGG
SGGGGSQVNLLQSGAALVKPGASVKLSCKASGYTFTDYYIFWVKQSHGKSLEWIGYI
NPNSGSTNYNEKFKRKATLSVDKSTNTAYMELSRLTSEDSATYYCTRRAYYGYNPFD
YWGQGVMVTVSS
CD318 specific CAR sequence VH-linker-VL
SEQ ID NO: 29
EVQLQQSGAELVRPGALVKLSCKASGFNIKDYYIHWVKQRPEQGLEWIGWIDPENG
HTIYDPKFQGKASITADTSSNTAYLQLSSLTSEDTAVYYCARLTGTTYAMDYWGQGT
SVTVSSGGGGSGGGGSGGGGSDIVMTQSHKFMSTSVGDRVSITCKASQDVSTAVAW
YQQKSGQSPKLLIYWASTRHTGVPDRFTGSGSGTDYTLTISSVQAEDLALYYCQQHY
STPYTFGGGTKLEIK
CD318 specific CAR sequence VL-linker-VH
SEQ ID NO: 30
DIVMTQSHKFMSTSVGDRVSITCKASQDVSTAVAWYQQKSGQSPKLLIYWASTRHT
GVPDRFTGSGSGTDYTLTISSVQAEDLALYYCQQHYSTPYTFGGGTKLEIKGGGGSG
GGGSGGGGSEVQLQQSGAELVRPGALVKLSCKASGFNIKDYYIHWVKQRPEQGLEW
IGWIDPENGHTIYDPKFQGKASITADTSSNTAYLQLSSLTSEDTAVYYCARLTGTTYA
MDYWGQGTSVTVSS
TSPAN8 specific CAR sequence VH-linker-VL
SEQ ID NO: 31
EVKLLESGGGLVQPGGSMRLSCAASGFTFTDFYMNWIRQPAGKAPEWLGFIRNKAS
GYTTEYNPSVKGRFTISRDNTQNMLYLQMNTLRAEDTATYYCARAHSYYGYDYFDY
WGQGVMVTVSSGGGGSGGGGSGGGGSDIQMTQSPASLSASLEEIVTITCQASQDIGN
WLSWYQQKPGKSPQLLIYGATSLADGVPSRFSGSRSGTQYSLKISRLQVEDIRIYYCL
QAYSAPWTFGGGTKLELK
TSPAN8 specific CAR sequence VL-linker-VH
SEQ ID NO: 32
DIQMTQSPASLSASLEEIVTITCQASQDIGNWLSWYQQKPGKSPQLLIYGATSLADGV
PSRFSGSRSGTQYSLKISRLQVEDIRIYYCLQAYSAPWTFGGGTKLELKGGGGSGGGG
SGGGGSEVKLLESGGGLVQPGGSMRLSCAASGFTFTDFYMNWIRQPAGKAPEWLGFI
RNKASGYTTEYNPSVKGRFTISRDNTQNMLYLQMNTLRAEDTATYYCARAHSYYGY
DYFDYWGQGVMVTVSS
MSLN specific CAR sequence 1 VH-linker-VL
SEQ ID NO: 33
EVQLVQSGGGLVQPGGSLRLSCAASGFTFDDYAMHWVRQAPGKGLEWVSGISWNS
GSIGYADSVKGRFTISRDNAKNSLYLQMNSLRAEDTALYYCAKDLSSVAGPFNYWG
QGTLVTVSSGGGGSGGGGSGGGGSSSELTQDPAVSVALGQTVRITCQGDSLRSYYAS
WYQQKPGQAPVLVIYGKNNRPSGIPDRFSGSSSGNTASLTITGAQAEDEADYYCNSR
DSSGNHLVFGGGTQLTVLG
MSLN specific CAR sequence 1 VL-linker-VH
SEQ ID NO: 34
SSELTQDPAVSVALGQTVRITCQGDSLRSYYASWYQQKPGQAPVLVIYGKNNRPSGI
PDRFSGSSSGNTASLTITGAQAEDEADYYCNSRDSSGNHLVFGGGTQLTVLGGGGGS
GGGGSGGGGSEVQLVQSGGGLVQPGGSLRLSCAASGFTFDDYAMHWVRQAPGKGL
EWVSGISWNSGSIGYADSVKGRFTISRDNAKNSLYLQMNSLRAEDTALYYCAKDLSS
VAGPFNYWGQGTLVTVSS
MSLN specific CAR sequence 1 VH
SEQ ID NO: 35
EVQLVQSGGGLVQPGGSLRLSCAASGFTFDDYAMHWVRQAPGKGLEWVSGISWNS
GSIGYADSVKGRFTISRDNAKNSLYLQMNSLRAEDTALYYCAKDLSSVAGPFNYWG
QGTLVTVSS
MSLN specific CAR sequence 1 VH-linker-VL
SEQ ID NO: 36
SSELTQDPAVSVALGQTVRITCQGDSLRSYYASWYQQKPGQAPVLVIYGKNNRPSGI
PDRFSGSSSGNTASLTITGAQAEDEADYYCNSRDSSGNHLVFGGGTQLTVLG
MSLN specific CAR sequence 2 VH-linker-VL
SEQ ID NO: 37
EVQLVQSGGGLVQPGRSQRLSCAASGFTFDDYAMHWVRQAPGKGLEWVSGISWNS
GSIGYADSVKGRFTISRDNAKNSLYLQMNSLRAEDTALYYCAKDISSSAGNAFDIWG
QGTMVTVSSGGGGSGGGGSGGGGSSSELTQDPAVSVALGQTVRITCQGDRLRSYYA
SWYQQKPGQAPVLVIYGKNNRPSGIPDRFSGSDSGDTASLTITGAQAEDEADYYCHS
RDSGGNHVVFGGGTQLTVLG
MSLN specific CAR sequence 2 VH-linker-VL
SEQ ID NO: 38
SSELTQDPAVSVALGQTVRITCQGDRLRSYYASWYQQKPGQAPVLVIYGKNNRPSGI
PDRFSGSDSGDTASLTITGAQAEDEADYYCHSRDSGGNHVVFGGGTQLTVLGSGGG
GSGGGGSGGGGSEVQLVQSGGGLVQPGRSQRLSCAASGFTFDDYAMHWVRQAPGK
GLEWVSGISWNSGSIGYADSVKGRFTISRDNAKNSLYLQMNSLRAEDTALYYCAKDI
SSSAGNAFDIWGQGTMVTVS
MSLN specific CAR sequence 2 VH
SEQ ID NO: 39
EVQLVQSGGGLVQPGRSQRLSCAASGFTFDDYAMHWVRQAPGKGLEWVSGISWNS
GSIGYADSVKGRFTISRDNAKNSLYLQMNSLRAEDTALYYCAKDISSSAGNAFDIWG
QGTMVTVSS
MSLN specific CAR sequence 2 VH-linker-VL
SEQ ID NO: 40
SSELTQDPAVSVALGQTVRITCQGDRLRSYYASWYQQKPGQAPVLVIYGKNNRPSGI
PDRFSGSDSGDTASLTITGAQAEDEADYYCHSRDSGGNHVVFGGGTQLTVLGS

### EXAMPLES

A so called adapter CAR (Ad CAR) was produced according to WO2018078066A1. Such Ad CARs are specific to an adapter that in turn is specific to a target. In the present case, the Ad CAR T cell is specific for LC-LC-Biotin and the adapters are biotinylated antibodies (ABs) specific for the four targets CD66c, CD318, TSPAN8, CLA and Mesothelin.

### Experiment

As biotinylated antibodies (Abs) for the adapters were used (all obtainable from Miltenyi Biotec B.V. & Co. K):
CD66c: Clone REA414
CD318: Clone REA194
TSPAN8: Clone REA443
Mesothelin (MSLN): Clone REA1057

CAR T cells specific for LC-LC-Biotin were produced and frozen. The antibodies were obtained already biotinylated.

24 h Before the start of the assay CAR T cells were thawed and inoculated in TexMACS™ medium supplemented with 12.5 ng/ml IL-7 and IL-15.0. On the day of use, CAR T cells were plated in 96-well culture plates at a density of 5×10⁴ cells per well in 100 μl medium. GFP⁺ PaCa cell lines PanCa0201, Panc0203 and AsPC1 were resuspended in 100 μl medium and inoculated with the CAR T cells at a density of 5×10³ resulting in an E:T ratio (effector:target or CAR T cell:target cell) of 10:1. Biotinylated antibodies were diluted into 50 μl of medium to a concentration of 4000 ng/ml so that upon addition into the respective wells final antibody concentration was 1000 ng/ml. Plates were then kept in an Incucyte S3 device (EssenBiosciences, 37° C.+5% CO₂) for 42-44 h. At this point green object confluence (i.e. target cell confluence) was measured using the Incucyte S3. Measured values for each group were normalized to the green confluence at 0 h and to the respective unspecific effects caused by the target cells inoculated with Ad CAR T cells without adapter for each time point. The medium used in this assay was TexMACS™ Medium (Miltenyi Biotec B.V. & Co. KG) without supplements.

### Results

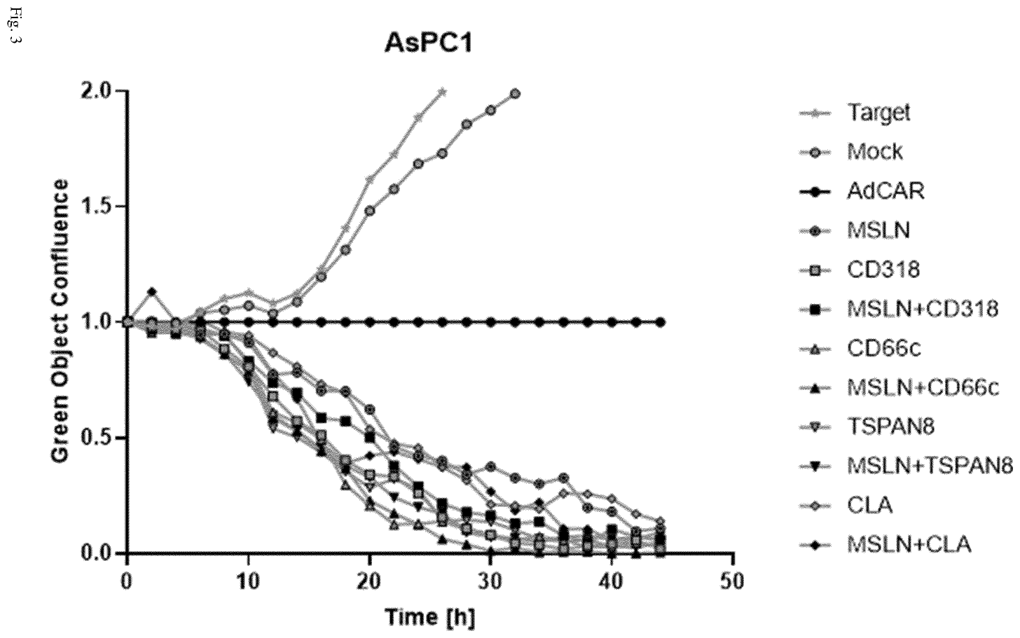

All target binders exhibit a clear specific killing over the control irrespective of binder and target cell as shown in FIGS. 3 to 5. The killing ranges from almost complete killing (group MSLN+CD66c on AsPC1 cells) to a lysis of around 40% of the target cells (group CLA on Panc0203).

FIG. 3 shows the results for different binders for CD66c, CD318, TSPAN8 and MSLN show all effective specific killing on AsPC1 cells. The figure shows the specific killing of Ad CAR T cells on the PaCa cell lines AsPC1 with different biotinylated binders. Wells contained 5×10⁴ Ad CAR T cells and 5×10³ PaCa target cells and 1000 ng/ml of the respective binder. Specific killing was assessed for 44 h of co-culture. Error bars are not depicted for clarity.

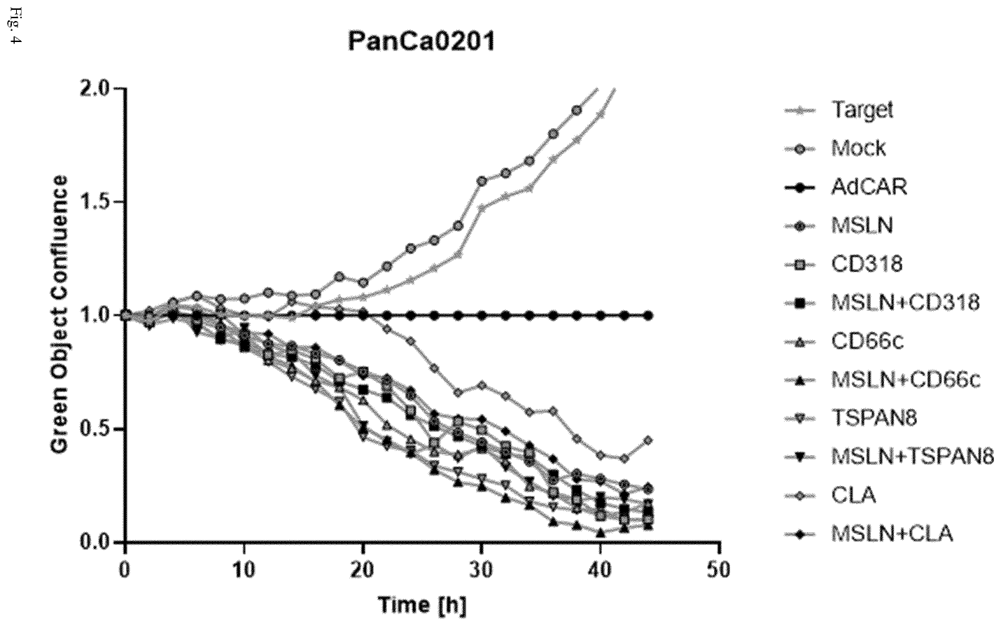

FIG. 4 shows the results for different binders for CD66c, CD318, TSPAN8 and MSLN show all effective specific killing on Panc0201 cells. The figure shows the specific killing of Ad CAR T cells on the PaCa cell lines Panc0201 with different biotinylated binders. Wells contained 5×10⁴ Ad CAR T cells and 5×10³ PaCa target cells and 1000 ng/ml of the respective binder. Specific killing was assessed for 44 h of co-culture. Error bars are not depicted for clarity.

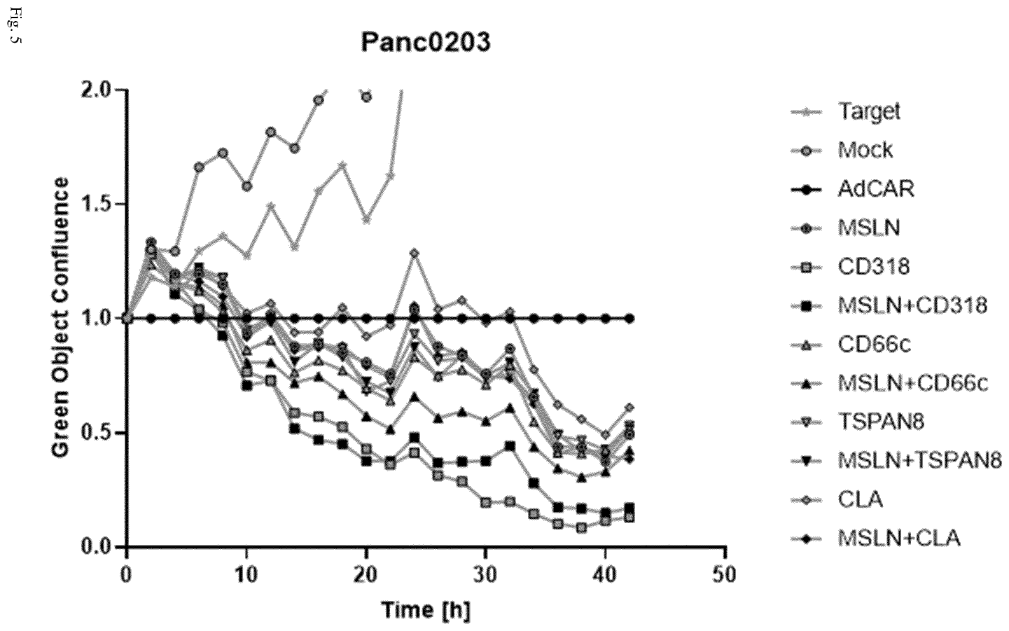

FIG. 5 shows the results for different binders for CD66c, CD318, TSPAN8 and MSLN show all effective specific killing on Panc0203 cells. The figure shows the specific killing of Ad CAR T cells on the PaCa cell lines Panc0203 with different biotinylated binders. Wells contained 5×10⁴ Ad CAR T cells and 5×10³ PaCa target cells and 1000 ng/ml of the respective binder. Specific killing was assessed for 4 h of co-culture. Error bars are not depicted for clarity.

In summary, it is shown on three independent cell lines for four different targets and combinations thereof that effective and specific killing of the target cells takes place with the binders according to the invention.

1. A chimeric antigen receptor (CAR), comprising an antigen binding domain specific for MSLN in combination with one or more antigen binding domains specific for an antigen selected from the group consisting of CLA, CD66c, TSPAN8 and CD318.

2. A chimeric antigen receptor (CAR) according to claim 1, characterized in that the CAR comprises an antigen binding domain specific for MSLN in combination with at least two antigen binding domains specific for at least two different antigens selected from the group consisting of CLA, CD66c, TSPAN8 and CD318.

3. A chimeric antigen receptor (CAR) according to claim 1 or 2, characterized in that the CAR comprises an antigen binding domain, a transmembrane domain and an intracellular signaling domain and wherein the intracellular signaling domain is triggered by binding of the antigen binding domain specific for MSLN or one antigen binding domain specific for an antigen selected from the group consisting of CLA, CD66c, TSPAN8 and CD318 to the appropriate antigens.

4. A chimeric antigen receptor (CAR) according to at least one of the claims 1 to 3, characterized in that the CAR comprises an antigen binding domain, an transmembrane domain and an intracellular signaling domain and comprising an antigen binding domain specific for MSLN in combination with one or more antigen binding domains specific for an antigen selected from the group consisting of CLA, CD66c, TSPAN8 and CD318 which are conjugated to the same or a different transmembrane domain and/or intracellular signaling domain.

5. A chimeric antigen receptor (CAR) according to at least one of the claims 1 to 4, characterized in that the transmembrane domain comprises a sequence of the transmembrane domains of 4-1BB, CD8 and/or CD28; and the intracellular signaling domain comprises a sequence of the intracellular signaling domains of one or more of CD28, CD137 and CD3zeta.

6. A chimeric antigen receptor (CAR) according to at least one of the claims 1 to 5, characterized in that in that the CAR comprises an anti-tag binding region which can bind to a tag which is coupled to an antigen binding domain specific for one or more antigens selected from the group consisting of MSLN, CLA, CD66c, TSPAN8 and CD318.

7. A chimeric antigen receptor (CAR) according to at least one of the claims 1 to 5, characterized in that in that the CAR comprises an anti-tag binding region which can bind to a tag which is coupled to an antigen binding domain specific for MSLN in combination with at least two antigen binding domains specific for at least two different antigens selected from the group consisting of CLA, CD66c, TSPAN8 and CD318.

8. Method of binding a cancer cell with a CAR according to at least one of the claims 1 to 7.

9. A population of engineered cells expressing at least one CAR according to at least one of the claims 1 to 7.

10. Use of the population of engineered cells according to claim 9 for treatment of human cancer.

11. A pharmaceutical composition comprising a population of engineered cells expressing a CAR according to at least one of the claims 1 to 7.

12. Use of the pharmaceutical composition according to claim 11 for treatment of human cancer.

13. Use of the pharmaceutical composition according to claim 11 in combination with a chemotherapeutic, radiation, or immunomodulatory agent for treatment of cancer.


SEQUENCE LISTING (40 sequences)

SEQ ID NO: 1 - protein, 117 aa, Homo sapiens
  CLA VH
EVQLVESGGGLVQPGNSLKLSCSASGFTFSSYGMHWIRQAPGEGLDWVAYISSSSGTVYADAVKARFTISRDNAKNTLYLQLNSLKSEDTAIYYCARAQNWDLFDYWGQGVMVTVSS

SEQ ID NO: 2 - protein, 112 aa, Homo sapiens
  CLA VL
QIMLTQQAESLWISPGERVSITCRASQSLLYTDGKHYLSWYQQKPGQTTKALIYHASVRTDGVPTRFIGSGSGTEFTLSIEHVQPEDFAIYYCLQTLKSPFTFGSGTKLEIK

SEQ ID NO: 3 - protein, 119 aa, Homo sapiens
  CD142 VH
QVQLKQSGPGLVQPSQSLSITCTVSGFSLSNYGVHWVRQSPGKGLEWLGVIWSGGSTDYNVAFISRLIITKDNSKSQVFLKMNSLQADDTAIYFCARTTGSVFNAMDHWGQGTSVTVSS

SEQ ID NO: 4 - protein, 106 aa, Homo sapiens
  CD142 VL
QIVLTQSPALMSASPGEKVTMTCSASSSVTYMYWYQQKPRSSPKPWIYLTSNLASGVPARFSGSGSGTSYSLTISSVEAEDAATYYCQQWSSNPLTFGAGTKLELK

SEQ ID NO: 5 - protein, 122 aa, Homo sapiens
  CD73 VH
EVQLQQSGAELVKPGASVKLSCTASGFNIKDTYIHWVKQRPEQGLEWIGRIDPATGNTEYDPKFQGKATITADTSSNTAYLHLSSLTSEDTAVYYCARGYYGSSYPPWFAYWGQGTLVTVSA

SEQ ID NO: 6 - protein, 107 aa, Homo sapiens
  CD73 VL
DIVMTQSHKFMSTSVGDRVSITCKASQDVGSAVAWYQQKPGQSPKLLIYWASTRHTGVPDRFTGSGSGTDFTLTISNVQSEDLADYFCQQYSSYPLTFGAGTKLELK

SEQ ID NO: 7 - protein, 117 aa, Homo sapiens
  CD49C VH
EVQLQQSGAELVKPGASVKLSCTASGFNIKDTYMHWVKQRPEQGLEWIGRIDPANGHTKYDPKFQGKATITADTSSNAAYLQLNSLTSEDTAVYYCARRVAYAMDYWGQGTSVTVSS

SEQ ID NO: 8 - protein, 106 aa, Homo sapiens
  CD49C VL
ENVLTQSPAIMSASPGEKVTMTCSASSSVTYMHWYQQKSSTSPKLWIYDTSKLASGVPGRFSGSGSGNSYSLTISSMEAEDVATYCCFQGSGYPLTFGGGTKLEIK

SEQ ID NO: 9 - protein, 118 aa, Homo sapiens
  CD66C VH
QVTLKESGPGILKPSQTLSLTCSFSGFSLSTSGMGVGWIRQPSGKSLEWLAHIWWNDERYYNPSLKNQLTISKDTSRNQVFLKITSVDTADTATYYCARSPRGYFDYWGHGTTLTVSS

SEQ ID NO: 10 - protein, 107 aa, Homo sapiens
  CD66C VL
DIVMTQSQKFMSTSVGDRVSVTCKASQNVVTNVAWYQQTPGQSPKALIYSASYRYSGVPDRFSGSGSGTDFTLTISNVQSGDLAEYFCQQYNSYPLTFGAGTKLELK

SEQ ID NO: 11 - protein, 120 aa, Homo sapiens
  CD104 VH
QVNLLQSGAALVKPGASVKLSCKASGYTFTDYYIFWVKQSHGKSLEWIGYINPNSGSTNYNEKFKRKATLSVDKSTNTAYMELSRLTSEDSATYYCTRRAYYGYNPFDYWGQGVMVTVSS

SEQ ID NO: 12 - protein, 107 aa, Homo sapiens
  CD104 VL
DIQMTQTPSSMPASLGERVTISCRASRGINNYLSWYQQNLDGTIKPLIYYTSNLQSGVPSRFSGSGSGTDYSLTISSLEPEDFAMYYCQQYDSSPWTFGGGTKLELK

SEQ ID NO: 13 - protein, 119 aa, Homo sapiens
  CD318 VH
EVQLQQSGAELVRPGALVKLSCKASGFNIKDYYIHWVKQRPEQGLEWIGWIDPENGHTIYDPKFQGKASITADTSSNTAYLQLSSLTSEDTAVYYCARLTGTTYAMDYWGQGTSVTVSS

SEQ ID NO: 14 - protein, 107 aa, Homo sapiens
  CD318 VL
DIVMTQSHKFMSTSVGDRVSITCKASQDVSTAVAWYQQKSGQSPKLLIYWASTRHTGVPDRFTGSGSGTDYTLTISSVQAEDLALYYCQQHYSTPYTFGGGTKLEIK

SEQ ID NO: 15 - protein, 123 aa, Homo sapiens
  TSPAN8 VH
EVKLLESGGGLVQPGGSMRLSCAASGFTFTDFYMNWIRQPAGKAPEWLGFIRNKASGYTTEYNPSVKGRFTISRDNTQNMLYLQMNTLRAEDTATYYCARAHSYYGYDYFDYWGQGVMVTVSS

SEQ ID NO: 16 - protein, 107 aa, Homo sapiens
  TSPAN8 VL
DIQMTQSPASLSASLEEIVTITCQASQDIGNWLSWYQQKPGKSPQLLIYGATSLADGVPSRFSGSRSGTQYSLKISRLQVEDIRIYYCLQAYSAPWTFGGGTKLELK

SEQ ID NO: 17 - protein, 244 aa, Homo sapiens
  CLA SPECIFIC SCFV VH-LINKER-VL
EVQLVESGGGLVQPGNSLKLSCSASGFTFSSYGMHWIRQAPGEGLDWVAYISSSSGTVYADAVKARFTISRDNAKNTLYLQLNSLKSEDTAIYYCARAQNWDLFDYWGQGVMVTVSSGGGGSGGGGSGGGGSQIMLTQQAESLWISPGERVSITCRASQSLLYTDGKHYLSWYQQKPGQTTKALIYHASVRTDGVPTRFIGSGSGTEFTLSIEHVQPEDFAIYYCLQTLKSPFTFGSGTKLEIK

SEQ ID NO: 18 - protein, 244 aa, Homo sapiens
  CLA SPECIFIC SCFV VL-LINKER-VH
QIMLTQQAESLWISPGERVSITCRASQSLLYTDGKHYLSWYQQKPGQTTKALIYHASVRTDGVPTRFIGSGSGTEFTLSIEHVQPEDFAIYYCLQTLKSPFTFGSGTKLEIKGGGGSGGGGSGGGGSEVQLVESGGGLVQPGNSLKLSCSASGFTFSSYGMHWIRQAPGEGLDWVAYISSSSGTVYADAVKARFTISRDNAKNTLYLQLNSLKSEDTAIYYCARAQNWDLFDYWGQGVMVTVSS

SEQ ID NO: 19 - protein, 240 aa, Homo sapiens
  CD142 SPECIFIC CAR SEQUENCE VH-LINKER-VL
QVQLKQSGPGLVQPSQSLSITCTVSGFSLSNYGVHWVRQSPGKGLEWLGVIWSGGSTDYNVAFISRLIITKDNSKSQVFLKMNSLQADDTAIYFCARTTGSVFNAMDHWGQGTSVTVSSGGGGSGGGGSGGGGSQIVLTQSPALMSASPGEKVTMTCSASSSVTYMYWYQQKPRSSPKPWIYLTSNLASGVPARFSGSGSGTSYSLTISSVEAEDAATYYCQQWSSNPLTFGAGTKLELK

SEQ ID NO: 20 - protein, 240 aa, Homo sapiens
  CD142 SPECIFIC CAR SEQUENCE VL-LINKER-VH
QIVLTQSPALMSASPGEKVTMTCSASSSVTYMYWYQQKPRSSPKPWIYLTSNLASGVPARFSGSGSGTSYSLTISSVEAEDAATYYCQQWSSNPLTFGAGTKLELKGGGGSGGGGSGGGGSQVQLKQSGPGLVQPSQSLSITCTVSGFSLSNYGVHWVRQSPGKGLEWLGVIWSGGSTDYNVAFISRLIITKDNSKSQVFLKMNSLQADDTAIYFCARTTGSVFNAMDHWGQGTSVTVSS

SEQ ID NO: 21 - protein, 244 aa, Homo sapiens
  CD73 SPECIFIC CAR SEQUENCE VH-LINKER-VL
EVQLQQSGAELVKPGASVKLSCTASGFNIKDTYIHWVKQRPEQGLEWIGRIDPATGNTEYDPKFQGKATITADTSSNTAYLHLSSLTSEDTAVYYCARGYYGSSYPPWFAYWGQGTLVTVSAGGGGSGGGGSGGGGSDIVMTQSHKFMSTSVGDRVSITCKASQDVGSAVAWYQQKPGQSPKLLIYWASTRHTGVPDRFTGSGSGTDFTLTISNVQSEDLADYFCQQYSSYPLTFGAGTKLELK

SEQ ID NO: 22 - protein, 244 aa, Homo sapiens
  CD73 SPECIFIC CAR SEQUENCE VL-LINKER-VH
DIVMTQSHKFMSTSVGDRVSITCKASQDVGSAVAWYQQKPGQSPKLLIYWASTRHTGVPDRFTGSGSGTDFTLTISNVQSEDLADYFCQQYSSYPLTFGAGTKLELKGGGGSGGGGSGGGGSEVQLQQSGAELVKPGASVKLSCTASGFNIKDTYIHWVKQRPEQGLEWIGRIDPATGNTEYDPKFQGKATITADTSSNTAYLHLSSLTSEDTAVYYCARGYYGSSYPPWFAYWGQGTLVTVSA

SEQ ID NO: 23 - protein, 238 aa, Homo sapiens
  CD49C SPECIFIC CAR SEQUENCE VH-LINKER-VL
EVQLQQSGAELVKPGASVKLSCTASGFNIKDTYMHWVKQRPEQGLEWIGRIDPANGHTKYDPKFQGKATITADTSSNAAYLQLNSLTSEDTAVYYCARRVAYAMDYWGQGTSVTVSSGGGGSGGGGSGGGGSENVLTQSPAIMSASPGEKVTMTCSASSSVTYMHWYQQKSSTSPKLWIYDTSKLASGVPGRFSGSGSGNSYSLTISSMEAEDVATYCCFQGSGYPLTFGGGTKLEIK

SEQ ID NO: 24 - protein, 238 aa, Homo sapiens
  CD49C SPECIFIC CAR SEQUENCE VL-LINKER-VH
ENVLTQSPAIMSASPGEKVTMTCSASSSVTYMHWYQQKSSTSPKLWIYDTSKLASGVPGRFSGSGSGNSYSLTISSMEAEDVATYCCFQGSGYPLTFGGGTKLEIKGGGGSGGGGSGGGGSEVQLQQSGAELVKPGASVKLSCTASGFNIKDTYMHWVKQRPEQGLEWIGRIDPANGHTKYDPKFQGKATITADTSSNAAYLQLNSLTSEDTAVYYCARRVAYAMDYWGQGTSVTVSS

SEQ ID NO: 25 - protein, 240 aa, Homo sapiens
  CD66C SPECIFIC CAR SEQUENCE VH-LINKER-VL
QVTLKESGPGILKPSQTLSLTCSFSGFSLSTSGMGVGWIRQPSGKSLEWLAHIWWNDERYYNPSLKNQLTISKDTSRNQVFLKITSVDTADTATYYCARSPRGYFDYWGHGTTLTVSSGGGGSGGGGSGGGGSDIVMTQSQKFMSTSVGDRVSVTCKASQNVVTNVAWYQQTPGQSPKALIYSASYRYSGVPDRFSGSGSGTDFTLTISNVQSGDLAEYFCQQYNSYPLTFGAGTKLELK

SEQ ID NO: 26 - protein, 240 aa, Homo sapiens
  CD66C SPECIFIC CAR SEQUENCE VL-LINKER-VH
DIVMTQSQKFMSTSVGDRVSVTCKASQNVVTNVAWYQQTPGQSPKALIYSASYRYSGVPDRFSGSGSGTDFTLTISNVQSGDLAEYFCQQYNSYPLTFGAGTKLELKGGGGSGGGGSGGGGSQVTLKESGPGILKPSQTLSLTCSFSGFSLSTSGMGVGWIRQPSGKSLEWLAHIWWNDERYYNPSLKNQLTISKDTSRNQVFLKITSVDTADTATYYCARSPRGYFDYWGHGTTLTVSS

SEQ ID NO: 27 - protein, 242 aa, Homo sapiens
  CD104 SPECIFIC CAR SEQUENCE VH-LINKER-VL
QVNLLQSGAALVKPGASVKLSCKASGYTFTDYYIFWVKQSHGKSLEWIGYINPNSGSTNYNEKFKRKATLSVDKSTNTAYMELSRLTSEDSATYYCTRRAYYGYNPFDYWGQGVMVTVSSGGGGSGGGGSGGGGSDIQMTQTPSSMPASLGERVTISCRASRGINNYLSWYQQNLDGTIKPLIYYTSNLQSGVPSRFSGSGSGTDYSLTISSLEPEDFAMYYCQQYDSSPWTFGGGTKLELK

SEQ ID NO: 28 - protein, 242 aa, Homo sapiens
  CD104 SPECIFIC CAR SEQUENCE VL-LINKER-VH
DIQMTQTPSSMPASLGERVTISCRASRGINNYLSWYQQNLDGTIKPLIYYTSNLQSGVPSRFSGSGSGTDYSLTISSLEPEDFAMYYCQQYDSSPWTFGGGTKLELKGGGGSGGGGSGGGGSQVNLLQSGAALVKPGASVKLSCKASGYTFTDYYIFWVKQSHGKSLEWIGYINPNSGSTNYNEKFKRKATLSVDKSTNTAYMELSRLTSEDSATYYCTRRAYYGYNPFDYWGQGVMVTVSS

SEQ ID NO: 29 - protein, 241 aa, Homo sapiens
  CD318 SPECIFIC CAR SEQUENCE VH-LINKER-VL
EVQLQQSGAELVRPGALVKLSCKASGFNIKDYYIHWVKQRPEQGLEWIGWIDPENGHTIYDPKFQGKASITADTSSNTAYLQLSSLTSEDTAVYYCARLTGTTYAMDYWGQGTSVTVSSGGGGSGGGGSGGGGSDIVMTQSHKFMSTSVGDRVSITCKASQDVSTAVAWYQQKSGQSPKLLIYWASTRHTGVPDRFTGSGSGTDYTLTISSVQAEDLALYYCQQHYSTPYTFGGGTKLEIK

SEQ ID NO: 30 - protein, 241 aa, Homo sapiens
  CD318 SPECIFIC CAR SEQUENCE VL-LINKER-VH
DIVMTQSHKFMSTSVGDRVSITCKASQDVSTAVAWYQQKSGQSPKLLIYWASTRHTGVPDRFTGSGSGTDYTLTISSVQAEDLALYYCQQHYSTPYTFGGGTKLEIKGGGGSGGGGSGGGGSEVQLQQSGAELVRPGALVKLSCKASGFNIKDYYIHWVKQRPEQGLEWIGWIDPENGHTIYDPKFQGKASITADTSSNTAYLQLSSLTSEDTAVYYCARLTGTTYAMDYWGQGTSVTVSS

SEQ ID NO: 31 - protein, 245 aa, Homo sapiens
  TSPAN8 SPECIFIC CAR SEQUENCE VH-LINKER-VL
EVKLLESGGGLVQPGGSMRLSCAASGFTFTDFYMNWIRQPAGKAPEWLGFIRNKASGYTTEYNPSVKGRFTISRDNTQNMLYLQMNTLRAEDTATYYCARAHSYYGYDYFDYWGQGVMVTVSSGGGGSGGGGSGGGGSDIQMTQSPASLSASLEEIVTITCQASQDIGNWLSWYQQKPGKSPQLLIYGATSLADGVPSRFSGSRSGTQYSLKISRLQVEDIRIYYCLQAYSAPWTFGGGTKLELK

SEQ ID NO: 32 - protein, 245 aa, Homo sapiens
  TSPAN8 SPECIFIC CAR SEQUENCE VL-LINKER-VH
DIQMTQSPASLSASLEEIVTITCQASQDIGNWLSWYQQKPGKSPQLLIYGATSLADGVPSRFSGSRSGTQYSLKISRLQVEDIRIYYCLQAYSAPWTFGGGTKLELKGGGGSGGGGSGGGGSEVKLLESGGGLVQPGGSMRLSCAASGFTFTDFYMNWIRQPAGKAPEWLGFIRNKASGYTTEYNPSVKGRFTISRDNTQNMLYLQMNTLRAEDTATYYCARAHSYYGYDYFDYWGQGVMVTVSS

SEQ ID NO: 33 - protein, 244 aa, Homo sapiens
EVQLVQSGGGLVQPGGSLRLSCAASGFTFDDYAMHWVRQAPGKGLEWVSGISWNSGSIGYADSVKGRFTISRDNAKNSLYLQMNSLRAEDTALYYCAKDLSSVAGPFNYWGQGTLVTVSSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNHLVFGGGTQLTVLG

SEQ ID NO: 34 - protein, 244 aa, Homo sapiens
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHLVFGGGTQLTVLGGGGGSGGGGSGGGGSEVQLVQSGGGLVQPGGSLRLSCAASGFTFDDYAMHWVRQAPGKGLEWVSGISWNSGSIGYADSVKGRFTISRDNAKNSLYLQMNSLRAEDTALYYCAKDLSSVAGPFNYWGQGTLVTVSS

SEQ ID NO: 35 - protein, 120 aa, Homo sapiens
EVQLVQSGGGLVQPGGSLRLSCAASGFTFDDYAMHWVRQAPGKGLEWVSGISWNSGSIGYADSVKGRFTISRDNAKNSLYLQMNSLRAEDTALYYCAKDLSSVAGPFNYWGQGTLVTVSS

SEQ ID NO: 36 - protein, 109 aa, Homo sapiens
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHLVFGGGTQLTVLG

SEQ ID NO: 37 - protein, 245 aa, Homo sapiens
EVQLVQSGGGLVQPGRSQRLSCAASGFTFDDYAMHWVRQAPGKGLEWVSGISWNSGSIGYADSVKGRFTISRDNAKNSLYLQMNSLRAEDTALYYCAKDISSSAGNAFDIWGQGTMVTVSSGGGGSGGGGSGGGGSSSELTQDPAVSVALGQTVRITCQGDRLRSYYASWYQQKPGQAPVLVIYGKNNRPSGIPDRFSGSDSGDTASLTITGAQAEDEADYYCHSRDSGGNHVVFGGGTQLTVLG

SEQ ID NO: 38 - protein, 245 aa, Homo sapiens
SSELTQDPAVSVALGQTVRITCQGDRLRSYYASWYQQKPGQAPVLVIYGKNNRPSGIPDRFSGSDSGDTASLTITGAQAEDEADYYCHSRDSGGNHVVFGGGTQLTVLGSGGGGSGGGGSGGGGSEVQLVQSGGGLVQPGRSQRLSCAASGFTFDDYAMHWVRQAPGKGLEWVSGISWNSGSIGYADSVKGRFTISRDNAKNSLYLQMNSLRAEDTALYYCAKDISSSAGNAFDIWGQGTMVTVS

SEQ ID NO: 39 - protein, 121 aa, Homo sapiens
EVQLVQSGGGLVQPGRSQRLSCAASGFTFDDYAMHWVRQAPGKGLEWVSGISWNSGSIGYADSVKGRFTISRDNAKNSLYLQMNSLRAEDTALYYCAKDISSSAGNAFDIWGQGTMVTVSS

SEQ ID NO: 40 - protein, 110 aa, Homo sapiens
SSELTQDPAVSVALGQTVRITCQGDRLRSYYASWYQQKPGQAPVLVIYGKNNRPSGIPDRFSGSDSGDTASLTITGAQAEDEADYYCHSRDSGGNHVVFGGGTQLTVLGS